(19) United States
(12) Patent Application Publication
(10) Pub. No.: US 2026/0256865 A1
(43) Pub. Date: Sep. 3, 2026

(54) PHARMACEUTICAL COMPOSITION FOR PREVENTING OR TREATING ALZHEIMER’S DISEASE, COMPRISING, AS ACTIVE INGREDIENT, LACTIC ACID BACTERIA FERMENTATION SOLUTION OF MORI FRUCTUS EXTRACT

(71) Applicants: DONGGUK UNIVERSITY WISE CAMPUS INDUSTRY-ACADEMY COOPERATION FOUNDATION, Gyeongsangbuk-do (KR); DONGGUK UNIVERSITY INDUSTRY-ACADEMY COOPERATION FOUNDATION, Seoul (KR)

(72) Inventors: Byung Soo KOO, Gyeonggi-do (KR); Kwang Ki KIM, Gyeonggi-do (KR); Quanfeng LIU, Gyeonggi-do (KR); Man Gi KIM, Gyeonggi-do (KR); Seok Seong KANG, Seoul (KR)

(73) Assignees: DONGGUK UNIVERSITY WISE CAMPUS INDUSTRY-ACADEMY COOPERATION FOUNDATION, Gyeongsangbuk-do (KR); DONGGUK UNIVERSITY INDUSTRY-ACADEMY COOPERATION FOUNDATION, Seoul (KR)

(21) Appl. No.: 18/846,820

(22) PCT Filed: Mar. 17, 2023
(86) PCT No.: PCT/KR2023/003558

§ 371 Date: Oct. 30, 2024

(30) Foreign Application Priority Data
Mar. 17, 2022 (KR) 10-2022-0033330

(51) Int. Cl. A61K 36/605; A61K 31/445; A61P 25/28; C12N 1/205; C12R 1/24

(52) U.S. Cl. CPC A61K 36/605; A61K 31/445; A61P 25/28; C12N 1/205; A61K 2236/19; A61K 2236/331; A61K 2236/333; A61K 2236/37; C12R 2001/24

(57) ABSTRACT

The present invention relates to a pharmaceutical composition for preventing or treating Alzheimer's disease, comprising, as an active ingredient, a mulberry fruit extract fermented with lactic acid bacteria. The mulberry fruit extract fermented with lactic acid bacteria improves memory and cognitive function, and thus can exhibit excellent effects on preventing, alleviating or treating Alzheimer's disease.

### TECHNICAL FIELD

This application claims the benefit of the filing date of Korean Patent Application No. 10-2022-0033330 filed with the Korean Intellectual Property Office on Mar. 17, 2022, the entire contents of which are incorporated herein by reference.

The present invention relates to a pharmaceutical composition for preventing or treating Alzheimer's disease, comprising, as an active ingredient, a mulberry (Morus alba linnaeus) fruit extract fermented with lactic acid bacteria.

### BACKGROUND ART

The global societal cost of dementia was estimated at $604 billion in 2010 alone, accounting for about 1% of the global total. Alzheimer's disease (AD) is the most common type of dementia, in which nerve cells in the cerebral cortex die and the frontal and temporal lobes of the brain atrophy, causing language impairment and severe short-term memory loss as, which gradually worsen, causing serious problems in daily life. Therefore, dementia not only places a huge economic burden on patients and their family members, but also has enormous socio-economic impacts, and thus it is clear that treatment for Alzheimer's disease patients, whose number is increasing due to the aging of the world population, is an urgent task at the present day.

Clinically used therapeutic agents for Alzheimer's disease, known to date, are divided into cholinesterase inhibitors that inhibit the breakdown of acetylcholine, a neurotransmitter in the brain, and N-methyl-D-aspartate (NMDA) receptor antagonists that inhibit the excessive secretion of glutamate, which is known to damage nerve cells. Donepezil, a drug belonging to the cholinesterase inhibitor class, was developed by Eisai Pharmaceutical Company in Japan and approved by the US FDA in late 1996. Donepezil is sold in over 30 countries around the world and has been sold in Korea since 1999. However, as the efficacy and practicality of donepezil, which is most widely used for the treatment of Alzheimer's disease today, are falling short of expectations, studies have been conducted on the combined use or mixing of drugs to increase therapeutic effects against Alzheimer's disease and reduce side effects. In addition, due to increasing interest in preventive medicine that can prevent Alzheimer's disease pathology before the progression thereof, studies on non-pharmacological combination therapies are also ongoing. In addition, in academic circles, there has recently been a growing trend in studies focused on revealing the relationship between gut microbes and neurological diseases, and studies have been reported that indicate that gut microbes have a significant impact on each other through the immune system, intestinal mucosa, and neuropsychiatric interactions. Thus, studies on the relationship between gut microbes and Alzheimer's disease have attracted great interest.

Accordingly, the present inventors have sought to find a new composition derived from a natural substance that can alleviate Alzheimer's disease with fewer drug-induced side effects, and as a result, have developed a composition that may be applied to prevent, ameliorate or treat Alzheimer's disease by containing useful physiologically active substances extracted from mulberry fruit, a medicinal herb verified in oriental medicine, through a lactic acid bacteria fermentation method.

### DISCLOSURE

### Technical Problem

The technical problem to be solved by the present invention is to provide a composition that has few side effects and is capable of alleviating, preventing or treating Alzheimer's disease.

However, the technical problems to be solved by the present invention are not limited to the technical problems mentioned above, and other technical problems not mentioned will be clearly understood by those skilled in the art from the following description.

### Technical Solution

According to one aspect of the present invention, there is provided a pharmaceutical composition for preventing or treating Alzheimer's disease, comprising a mulberry fruit extract fermented with lactic acid bacteria.

According to another aspect of the present invention, there is provided a health functional food for preventing or alleviating Alzheimer's disease, comprising a mulberry fruit extract fermented with lactic acid bacteria.

### Advantageous Effects

A composition comprising a mulberry fruit extract fermented with lactic acid bacteria according to the present invention may improve memory and cognitive function, and thus exhibit excellent effects on the prevention, alleviation or treatment of Alzheimer's disease.

The effects of the present invention are not limited to the effects mentioned above, and effects not mentioned will be clearly understood by those skilled in the art from the specification of the present invention.

### BRIEF DESCRIPTION OF DRAWINGS

FIG. 1 shows the results of a passive avoidance test for mice in a normal group, a control group, a lactic acid bacteria-fermented mulberry fruit extract group (DF01), a lactic acid bacteria-fermented mulberry fruit extract+donepezil group (DF01+DON), and a donepezil group (DON).

FIG. 2 shows the results of a Morris water maze test for mice in a normal group, a control group, a lactic acid bacteria-fermented mulberry fruit extract group (DF01), a lactic acid bacteria-fermented mulberry fruit extract+donepezil group (DF01+DON), and a donepezil group (DON).

FIG. 3 shows the results of a Y-maze test for mice in a normal group, a control group, a lactic acid bacteria-fermented mulberry fruit extract group (DF01), a lactic acid bacteria-fermented mulberry fruit extract+donepezil group (DF01+DON), and a donepezil group (DON).

FIG. 4 shows the results of analyzing the expression levels of Aβ42, p-Tau, BACE and p-AMPKα proteins in mice in a normal group, a control group, a lactic acid bacteria-fermented mulberry fruit extract group (DF01), a lactic acid bacteria-fermented mulberry fruit extract+donepezil group (DF01+DON), and a donepezil group (DON).

FIG. 5 shows the results of analyzing the cell viability of neuronal cell lines treated with a mulberry fruit extract (MAL), a lactic acid bacteria-fermented mulberry fruit extract (DF01), donepezil (DON), lactic acid bacteria-fermented mulberry fruit extract+donepezil (DF01+DON), and amyloid-beta, respectively.

FIG. 6 shows the results of analyzing the reactive oxygen species levels in neuronal cell lines treated with a mulberry fruit extract (MAL), a lactic acid bacteria-fermented mulberry fruit extract (DF01), donepezil (DON), lactic acid bacteria-fermented mulberry fruit extract+donepezil (DF01+DON), and amyloid-beta, respectively.

### BEST MODE

Throughout the present specification, it is to be understood that when any part is referred to as “comprising” any component, it does not exclude other components, but may further comprise other components, unless otherwise specified.

Throughout the present specification, the units “parts by weight” and “wt %” may refer to the weight ratio between components.

Hereinafter, the present invention will be described in more detail.

According to one embodiment of the present invention, there is provided a pharmaceutical composition for preventing or treating Alzheimer's disease, comprising a mulberry fruit extract fermented with lactic acid bacteria.

The mulberry fruit is the fruit of Morus alba L., a deciduous tree belonging to the Moraceae family, and is known to contain sugars, tannic acid, vitamins, and fatty acids such as linoleic acid. These mulberry fruits are harvested when they turn from blue to purple and black as they ripen, and are known to be effective for dizziness, tinnitus, dry mouth, wasting-thirst, tonic, pain relief, insomnia, lumbago, constipation, etc., and may be used for food and medicinal purposes. Korean Patent Application Publication No. 10-2005-0067131 relates to a composition containing a mulberry fruit extract having monoamine oxidase (MAO) inhibitory activity, and discloses that the mulberry fruit extract is also effective for MAO-related diseases such as depression, Alzheimer's disease, and Parkinson's disease. Therefore, the present inventors selected mulberry fruit, which is effective in alleviating Alzheimer's disease, and could obtain a pharmaceutical composition for preventing or treating Alzheimer's disease, comprising, as an active ingredient, a mulberry fruit extract fermented with lactic acid bacteria, which is highly effective in alleviating Alzheimer's disease, by applying a lactic acid bacteria fermentation method to more efficiently utilize the active ingredients contained in the mulberry fruit.

In the present specification, “mulberry fruit extract” refers to an extract containing a useful physiologically active substance or active ingredient isolated from mulberry fruit, and when a solvent is used for extraction, refers to an extract containing the solvent together with the active ingredient isolated from mulberry fruit. The mulberry fruit may be in an unprocessed raw state, a dried state, or a combination thereof, and may be in a finely powdered state from which the active ingredient may be extracted efficiently. In addition, the mulberry fruit extract also includes an extract subjected to a conventional purification process. For example, the extract may also include fractions obtained by additionally performing various purification processes, such as separation using an ultrafiltration membrane having a certain molecular weight cut-off value, or separation by various chromatography systems (manufactured for separation according to size, charge, hydrophobicity or affinity). In addition, the mulberry fruit extract may be prepared in a powder form by additional processes such as reduced pressure distillation and freeze drying or spray drying.

According to one embodiment of the present invention, the mulberry fruit extract may be obtained by extraction using at least one method selected from a hot-water extraction method and an ultrasonic extraction method. In addition, the mulberry fruit extract may be obtained by extraction with at least one solvent selected from water and a C₁-C₄ alcohol.

The hot-water extraction method is useful for extracting water-soluble substances, and when it is performed using water or a C₁-C₄ alcohol as a solvent, it is simple, takes less time, and can reduce the possibility of solvent-induced toxicity, compared to when an organic solvent is used. The ultrasonic extraction method may increase extraction efficiency by destroying solids through the impact effect of ultrasonic energy. The mulberry fruit extract obtained by this method may include any one selected from among an extract obtained by extraction, a dilution or concentrate of the extract, a dried product obtained by drying the extract, and a crude or purified product thereof.

Although the mulberry fruit extract is preferably obtained by extraction using at least one method selected from the hot-water extraction method and the ultrasonic extraction method so that the active ingredients of mulberry fruit may be extracted to the maximum extent, any extraction method known in the art may be used without limitation. Examples of extraction methods known in the art include extraction by cold maceration, ultrasonic extraction, reflux cooling extraction, hot-water extraction, pressurized extraction, solvent extraction, supercritical extraction, ultrasonic extraction, etc.

In addition, as a solvent that is used for the extraction method known in the art, any extraction solvent known in the art may be used without limitation. Examples of extraction solvents include purified water, distilled water, C₁-C₄ alcohols, acetic acid, dichloromethane, dimethyl formamide, dimethyl sulfoxide (DMSO), acetone, acetonitrile, ethyl acetate, methyl acetate, pentane, hexane, chloroform, diethyl ether, carbon tetrachloride, tetrahydrofuran (THF), etc. Examples of the C₁-C₄ alcohols include methanol, ethanol, propanol, n-butanol, iso-butanol, etc.

In the present specification, “lactic acid bacteria” refers to total bacteria that degrade carbohydrates into lactic acid in their metabolic process.

In the present specification, “fermentation”, refers, in a narrow sense, to a sugar degradation process of obtaining energy without using oxygen, and, in a broad sense, to a process of obtaining substances useful to humans using microorganisms or fungi. In addition, the “extract fermented” includes a substance useful to humans, produced through various enzymatic actions by culturing lactic acid bacteria in a substance to be fermented, and refers to a product obtained by fermenting the mulberry fruit extract with lactic acid bacteria.

According to one embodiment of the present invention, the lactic acid bacteria may be at least one selected from Lactobacillus brevis DF01 and Pediococcus acidilactici K10. The lactic acid bacteria may be Lactobacillus and/or Pediococcus that are easy to handle and have excellent fermentation efficiency, preferably Lactobacillus brevis DF01 or Pediococcus acidilactici K10, more preferably Lactobacillus brevis DF01. The fermentation process using such lactic acid bacteria has an advantage over general biological fermentation or solvent-based extraction methods in that the extraction of active ingredients is easier.

According to one embodiment of the present invention, the mulberry fruit extract fermented with lactic acid bacteria may be obtained by inoculating and fermenting 100 parts by weight of the mulberry fruit extract with 0.1 to 5 parts by weight of lactic acid bacteria. The mulberry fruit extract fermented with lactic acid bacteria is obtained by inoculating the mulberry fruit extract with lactic acid bacteria and culturing the lactic acid bacteria under optimal growth conditions. In order to improve fermentation efficiency, it is preferable to inoculate 100 parts by weight of the mulberry fruit extract with 0.1 to 5 parts by weight of lactic acid bacteria.

According to one embodiment of the present invention, the mulberry fruit extract fermented with lactic acid bacteria may be obtained by fermentation at 20° C. to 30° C. for 2 to 5 days. When the fermentation temperature and fermentation time for obtaining the mulberry fruit extract fermented with lactic acid bacteria are within the above-described ranges, lactic acid bacteria may be cultured under optimal growth conditions, and the fermentation efficiency may be improved.

According to one embodiment of the present invention, the pharmaceutical composition may further comprise donepezil. When the mulberry fruit extract fermented with lactic acid bacteria according to the present invention is co-administered with donepezil, the effect of improving memory and cognitive function may be enhanced.

According to one embodiment of the present invention, the mulberry fruit extract fermented with lactic acid bacteria and donepezil may be administered at a weight ratio of 1:0.01 to 1:100. Specifically, the weight ratio between the mulberry fruit extract fermented with lactic acid bacteria and donepezil may be 1:0.01 to 1:100, 1:0.1 to 1:50, 1:1 to 1:20, 1:5 to 1:15, or 1:10. The mulberry fruit extract fermented with lactic acid bacteria and donepezil are administered at a weight ratio within the above-described range, the effect of improving memory and cognitive function may be enhanced.

In the present specification, “comprising, as an active ingredient” means containing in a dose range that brings about the effect of preventing, alleviating, or treating Alzheimer's disease. The dose range may vary depending on the severity of the disease and the formulation, and the number of applications may also vary depending on the age, weight, and constitution of the patient.

The pharmaceutical composition of the present invention is administered in a pharmaceutically effective amount.

In the present specification, “pharmaceutically effective amount” means an amount sufficient to treat a disease at a reasonable benefit/risk ratio applicable to any medical treatment or alleviation. The effective dose level may be determined depending on factors, including the kind and severity of the patient's disease, the patient's age and sex, the activity of the drug, sensitivity to the drug, the time of administration, the route of administration, excretion rate, the period of treatment, and drugs used in combination with the composition, as well as other factors well known in the medical field. For example, the effective amount includes an effective amount of 0.001 mg/kg to 100 mg/kg, 0.01 mg/kg to 10 mg/kg, or 0.1 mg/kg to 1 mg/kg. The upper limit of the effective amount of the pharmaceutical composition according to the present invention may be selected within an appropriate range by a person skilled in the art.

The pharmaceutical composition according to the present invention may comprise an effective amount of the mulberry fruit extract fermented with lactic acid bacteria alone or in combination with at least one pharmaceutically acceptable carrier, excipient or diluent.

The pharmaceutically acceptable carrier, excipient or diluent refers to a substance which is physiologically acceptable and, when administered to the human beings, generally does not cause allergic reactions such as gastrointestinal disorders and dizziness, or similar reactions. Examples of the carriers, excipients and diluents include, but are not limited to, lactose, dextrose, sucrose, sorbitol, mannitol, xylitol, erythritol, maltitol, starch, gum acacia, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil. In addition, the composition may further comprise a filler, an anticoagulant, a lubricant, a wetting agent, a fragrance, an emulsifier, and a preservative.

The mulberry fruit extract fermented with lactic acid bacteria may be administered in various oral and parenteral dosage forms.

Solid formulations for oral administration include tablets, pills, powders, granules, capsules and the like, and such solid formulations are prepared by mixing at least one compound with at least one excipient, for example, starch, calcium carbonate, sucrose, lactose or gelatin. In addition to simple excipients, lubricants such as magnesium stearate or talc are also be used. Liquid formulations for oral administration include suspensions, solutions, emulsions, and syrup, and may comprise various excipients, for example, wetting agents, flavoring agents, aromatics, and preservatives, in addition to water and liquid paraffin, which are frequently used simple diluents. Formulations for parenteral administration include sterilized aqueous solutions, non-aqueous solutions, suspensions, and emulsions. As non-aqueous solvents or suspending agents, propylene glycol, polyethylene glycol, vegetable oils such as olive oil, injectable esters such as ethyl oleate, and the like may be used.

Parenteral administration is by subcutaneous injection, intravenous injection, intramuscular injection, or intrathoracic injection or infusion. Here, in order to prepare a dosage form for parenteral administration, the mulberry fruit extract fermented with lactic acid bacteria may be mixed with a stabilizer or buffer in water to prepare a solution or suspension, which may then be prepared in an ampoule or vial unit dosage form. The composition may be sterilized and/or contain adjuvants such as preservatives, stabilizers, wetting agents or emulsifying agents, salts for regulating osmotic pressure, and/or buffers, and other therapeutically useful substances, and may be formulated by conventional methods such as mixing, granulation or coating.

In the present specification, “prevention” means preventing the occurrence of symptoms of Alzheimer's disease by administering, feeding, or applying the pharmaceutical composition or health functional food of the present invention to a subject not suffering from Alzheimer's disease to suppress or block the symptoms of Alzheimer's disease.

In the present specification, “treatment” includes completely curing the symptoms of Alzheimer's disease as well as partially curing, ameliorating and relieving the symptoms of Alzheimer's disease as a result of administering the pharmaceutical composition of the present invention to a subject suffering from Alzheimer's disease.

According to one embodiment of the present invention, there is provided a health functional food for preventing or alleviating Alzheimer's disease, comprising a mulberry fruit extract fermented with lactic acid bacteria.

In the present invention, the term “alleviation” is meant to include ameliorating or relieving the symptoms of Alzheimer's disease by administering, feeding, or applying the pharmaceutical composition or food composition of the present invention to a subject suffering from Alzheimer's disease.

In the present invention, the term “health functional food” refers to a food manufactured and processed using raw materials or ingredients that have functionality beneficial for the human body in compliance with the Health Functional Food Act, and the term “functionality” means that the intake of food is directed to controlling nutriments on the structure and function of the human body or achieving useful effects on health such as physiological effects. The health functional food or health supplement food of the present invention, obtained in this way, is very useful because it may be consumed on a daily basis.

The type of food is not particularly limited, and the term “food” includes all health functional foods in a conventional sense.

The matters mentioned with respect to the pharmaceutical composition and health functional food of the present invention are applied equally unless they contradict each other.

According to one embodiment of the present invention, there is provided a method for preventing or treating Alzheimer's disease, comprising a step of administering a mulberry fruit extract fermented with lactic acid bacteria to a subject in need thereof.

According to one embodiment of the present invention, there is provided the use of a mulberry fruit extract fermented with lactic acid bacteria in the manufacture of a medicament for preventing or treating Alzheimer's disease.

In the above method or use, the above detailed description of the pharmaceutical composition and health functional food may be applied.

According to one experimental example of the present invention, when the mulberry fruit extract fermented with lactic acid bacteria was administered to an animal model of Alzheimer's disease, memory and cognitive ability could be restored to a normal level, as demonstrated in behavioral tests such as a passive avoidance test, a Morris water maze test, and a Y-maze test. Therefore, a pharmaceutical composition comprising the mulberry fruit extract fermented with lactic acid bacteria may be useful for the prevention or treatment of Alzheimer's disease.

Method for Producing Mulberry Fruit Extract Fermented with Lactic Acid Bacteria

According to one embodiment of the present invention, there is provided a method for producing the pharmaceutical composition for preventing or treating Alzheimer's disease, comprising steps of: (a) powdering mulberry fruit; (b) extracting the powdered mulberry fruit with hot water at 80° C. to 120° C. for 1 to 5 hours to obtain a first extract; (c) ultrasonically extracting the first extract at 40° C. to 80° C. for 10 to 60 minutes to obtain a second extract; and (d) inoculating the second extract with lactic acid bacteria, followed by fermentation at 20° C. to 40° C. for 2 to 5 days to obtain a fermented extract.

Steps (a) to (d) will be described in detail below.

### Step (a) of Powdering Mulberry Fruit

The mulberry fruit may be in an unprocessed raw state, a dried state, or a combination thereof. In order to efficiently extract active ingredients from the mulberry fruit, the mulberry fruit is powdered.

As a method for powdering the mulberry fruit, any powdering method known in the art may be used without limitation. For example, the mulberry fruit may be powdered using a ball mill, a hammer mill, a rod mill, a vibration mill, a roll crusher, a centrifugal impact mill, a vertical bead mill, an attrition mill, a pin mill, a Micro pulverizer, crusher, a knife cutter, or the like.

Step (b) of Extracting Powdered Mulberry Fruit with Hot Water to Obtain First Extract

A first extract comprising the active ingredients of the mulberry fruit may be obtained by extracting the powdered mulberry fruit with hot water.

In the process of extracting the powdered mulberry fruit with hot water, it is preferable to use water as an extraction solvent in order to prevent a toxic organic solvent from remaining in the extract of this step or the fermented extract of a subsequent step or prevent unnecessary substances from being generated during the extraction process and to increase extraction efficiency. In the present invention, it is preferable to extract the powdered mulberry fruit with hot water at 80° C. to 120° C. for 1 to 5 hours. At this time, the powdered mulberry fruit and water may be mixed together at a ratio of 1:5 to 20 (w/v) and subjected to hot-water extraction. If the extraction condition in this step is lower than 80° C. or shorter than 1 hour, active ingredients may not be properly extracted from the mulberry fruit, and if the extraction condition is higher than 120° C. or longer than 5 hours, the active ingredients may be destroyed or lost due to heat. In addition, if the ratio of the mulberry fruit to water is less than 1:5, the active ingredients of the mulberry fruit may not be sufficiently extracted because the solvent is insufficient, and if the ratio is more than 1:20, the extracted active ingredients may be diluted in the solvent, and thus the extraction efficiency may be lowered and a further step of concentrating the extract may be required.

### Step (c) of Ultrasonically Extracting First Extract to Obtain Second Extract

The first extract may be subjected to ultrasonic treatment to obtain a second extract.

The first extract is a mixture of the solvent containing active ingredients extracted from the mulberry fruit and the solid mulberry fruit. In order to structurally stabilize the active ingredients in the solvent and prevent the active ingredients from being destroyed or lost by heat during ultrasonic treatment, the acidity of the first extract may be adjusted. At this time, it is preferable to adjust the pH of the first extract to 6 to 7 by adding an acidic or basic substance to the first extract. As the acidic or basic substance, any substance known in the art may be used without limitation.

The extraction method using ultrasonic waves is a method of performing extraction using energy generated by ultrasonic vibration. Ultrasonic waves can destroy an insoluble solvent contained in the mulberry fruit in an aqueous solvent, and the high local temperature generated at this time can increase the kinetic energy of reactant particles located in the vicinity, thus providing sufficient energy required for the reaction, and the impact effect of ultrasonic energy can induce high pressure, which promotes the mixing of the substance contained in the mulberry fruit and the solvent, thereby increasing the extraction efficiency. In the present invention, it is preferable to perform ultrasonically extract the first extract at 40° C. to 80° C. for 10 to 60 minutes. If the extraction condition in this step is lower than 40° C. or shorter than 10 minutes, the extraction efficiency may be low, and if the extraction condition is higher than 80° C. or longer than 30 minutes, active ingredients may be destroyed or lost due to the heat generated by the ultrasonic waves.

Step (d) of Inoculating and Fermenting Second Extract with Lactic Acid Bacteria to Obtain Fermented Extract

By fermenting the second extract with lactic acid bacteria, a fermented extract containing not only the active ingredients of the mulberry fruit but also products produced by the lactic acid bacteria using the same may be obtained.

The second extract is in a state in which the active ingredients of the mulberry fruit have been sufficiently extracted through the hot-water extraction and ultrasonic extraction steps. By fermenting these active ingredients of the mulberry fruit with lactic acid bacteria, the active ingredients may be converted into a form that is easier to absorb or utilize in the human body, or new useful substances may be produced through the fermentation.

As a method for fermenting the second extract with lactic acid bacteria, any lactic acid bacteria fermentation method known in the art may be used without limitation. In the present invention, in order to provide optimal culture conditions for the growth of lactic acid bacteria, it is preferable to inoculate the second extract with lactic acid bacteria, followed by fermentation at 20 to 40° C. for 2 to 5 days. In particular, it is more preferable to ferment the second extract at 25 to 35° C. If the fermentation temperature in this step is out of the above range, the fermentation rate may be decreased or unwanted fermentation products may be produced, due to the death of lactic acid bacteria, and if the fermentation time is out of the above range, the efficiency of fermentation may decrease or the raw material may deteriorate. The lactic acid bacteria may be cultured under both anaerobic and aerobic conditions, and thus both anaerobic fermentation and aerobic fermentation are possible. In addition, in order to promote the growth of the lactic acid bacteria, the second extract may be maintained at pH 4 to 4.5. In addition, in order to promote fermentation with lactic acid bacteria without significantly changing the efficacy of the second extract, a carbon source and energy source for lactic acid bacteria may be added to the second extract before inoculation and fermentation with lactic acid bacteria. In this case, the carbon source and an energy source may be added considering the fermentation time, fermentation degree, etc. As carbon source and energy source, any substances known in the art may be used without limitation, and examples thereof include oligosaccharides, lactose, glucose, fructose, sugar, and mixtures thereof.

The initial inoculation concentration of the lactic acid bacteria may be 10⁷ to 109 cfu/g, and 100 parts by weight of the second extract may be inoculated and fermented with 0.1 to 5 parts by weight of lactic acid bacteria. Here, if the initial inoculation concentration of lactic acid bacteria and the inoculation amount of lactic acid bacteria are out of the above ranges, fermentation may not occur properly and the production cost may increase.

The fermented extract obtained through this fermentation with lactic acid bacteria contains solids such as mulberry fruit residue, lactic acid bacteria, and fermentation impurities, which may form sediments or floating substances and have an adverse effect on the final product. Therefore, in order to remove the solids contained in the fermented extract, a step of filtering the fermented extract may be further performed. As a method for filtering the fermented extract, any filtration method known in the art may be used without limitation. For example, the fermented extract may be filtered using a filter press, a centrifuge, a filter paper, or the like. In order to completely remove the solids, the filtration process may be repeated one or more times.

In addition, the fermented extract may be dried to facilitate storage and use of the fermented extract. As a method for drying the fermented extract, any drying method known in the art may be used without limitation, and examples thereof include freeze drying, vacuum drying, ventilation drying, air blowing drying, hot-air drying, fluidized drying, spray drying, infrared drying, and high-frequency drying.

### MODE FOR INVENTION

Hereinafter, the present invention will be described in detail by way of examples and experimental examples. However, the examples and experimental examples according to the present invention may be modified in various different forms, and the scope of the present invention should not be interpreted as being limited to the examples and experimental examples described below. The examples and experimental examples of the present specification are provided to more completely explain the present invention to those of ordinary skill in the art.

### Example 1. Preparation of Mulberry Fruit Extract Fermented with Lactic Acid Bacteria

Mulberry (Morus alba) fruits were purchased from Taewondang Pharmaceutical Company (4^{th} floor, 50 Namsan-ro 7-gil, Jung-gu, Daegu, South Korea) and used. Mulberry fruits were powdered using a blender, extracted using distilled water at a ratio of 1:10 w/v in a constant-temperature water bath at 100° C. for 3 hours, and then further extracted using an ultrasonicator at 60° C. for 30 minutes. The extract was adjusted to pH 6.5 using 1 N NaOH, inoculated with 1 wt % of Lactobacillus brevis DF01 (1×10⁸ cfu/g), and fermented at 25° C. for 3 days. The fermented extract was filtered to remove any remaining bacteria and foreign substances, thereby preparing a mulberry fruit extract fermented with lactic acid bacteria.

Evaluation of Anti-Alzheimer's Disease Effect of Mulberry Fruit Extract Fermented with Lactic Acid Bacteria

### Animal Model of Alzheimer's Disease

As experimental animals, male two-month (20 to 26 g) transgenic mice (APPswe/PS1dE9 Tg) imported from Jackson Laboratory through Joongang Laboratory Animal Co., Ltd. were purchased and raised in the preclinical laboratory of Dongguk University Ilsan Hospital. The animals were maintained at a temperature of 22±1° C. and a humidity of 55±1% under 12-hour light-dark cycles and had access to food and water ad libitum.

Specifically, this APPswe/PS1dE9 (APP/PS1) mouse model contains two mutant genes (APP and PS1), which result in overexpression and deposition of amyloid-beta (Aβ, amyloid-β), rapid formation of amyloid plaques in the cerebral cortex and hippocampus at 4.5 months of age, and reduced neurogenesis around the dentate gyrus of the hippocampus. Therefore, this mouse model was used in the experiment.

The experiment was conducted with 7.5-month-old genetically modified mice for a total of 3 months. The passive avoidance test was performed once a month, and the Morris water maze test and the Y-maze test were performed 3 months after the start of the experiment.

Experimental animals were divided into five groups as follows:
Normal: a C57BL/6 (normal mouse) group (n=5) fed normal feed;
Control: an APP/PS1 (genetically modified mouse) group (n=5) fed normal feed;
Lactic acid bacteria-fermented mulberry fruit extract group (DFO1): an APP/PS1 (genetically modified mouse) group (n=5) fed lactic acid bacteria-fermented mulberry fruit extract (0.1 mg/kg/day) for 3 months;
Lactic acid bacteria-fermented mulberry fruit extract+donepezil group (DFO1+DON): an APP/PS1 (genetically modified mouse) group (n=5) fed lactic acid bacteria-fermented mulberry fruit extract (0.1 mg/kg/day) plus donepezil (1 mg/kg/day) for 3 months; and
Donepezil group (DON): an APP/PS1 (genetically modified mouse) group (n=5) fed donepezil (1 mg/kg/day) for 3 months.

### Experimental Example 1. Passive Avoidance Test

The memory of the mice was tested at 0, 1, 2, and 3 months after feeding.

### \textcircled{1} Passive Avoidance Test Apparatus

The test apparatus was a box (shuttle box, 53 cm W×44 cm H×33 cm D) containing two compartments. The guillotine door that acts as a door between the two compartments was positioned in the center of the box. One compartment had a very bright light bulb installed to create an environment that mice dislike, and the other compartment had a device that can apply an electric shock (scrambled foot-shock) to the entire floor (grid floor).

### \textcircled{2} Training Trial

Each mouse of the five experimental groups was placed in one compartment and allowed to explore for 15 seconds. Then, the mouse was stimulated with light and noise from the top of the box so that the mouse passed through the guillotine door and moved to the other quiet and dark compartment (electric shock chamber). When the mouse entered the electric shock chamber, the guillotine door automatically closed and an electric shock was applied. As the shock, a 0.3 mA current was applied for 3 seconds. After the mouse received the foot shock, the mouse was taken out of the box and placed back in the cage. Electric shock was applied to the mice of the experimental groups and the control group in the same manner. Mice that did not enter the shock chamber within 120 seconds were excluded from the experiment.

### \textcircled{3} Retention Trial

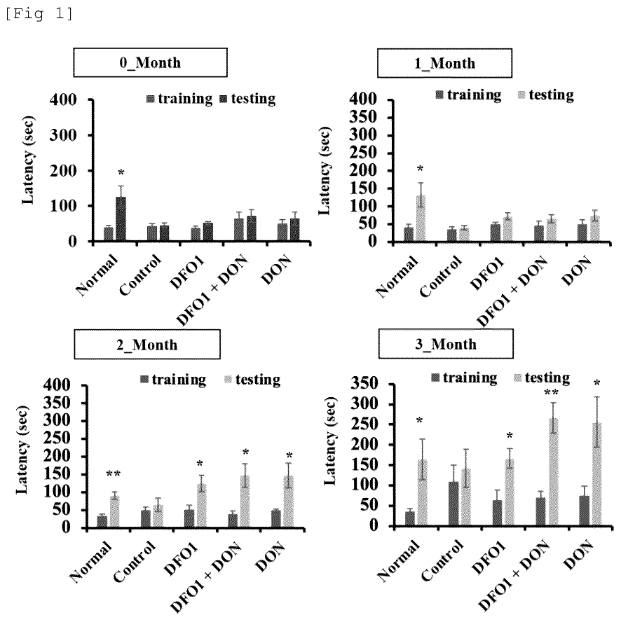

When a trained mouse receives an electric shock, it remembers the shock received on the previous day and avoids entering the electric shock chamber. As the time required to reach the electric shock chamber increases, it indicates that the passive-avoidance training and retention effect is better. 24 hours after the training, an electric shock was applied to the mouse, and then the step-through latency time to reach the shock chamber was measured up to 300 seconds (cut-off time). The results are shown in FIG. 1.

As shown in FIG. 1, in the control group on 2 months and 3 months, the testing trial time did not significantly increase compared to the training trial time. On the other hand, in the lactic acid bacteria-fermented mulberry fruit extract group (DFO1) fed the lactic acid bacteria-fermented mulberry fruit extract alone and the experimental group (DF01+DON) fed the lactic acid bacteria-fermented mulberry fruit extract and donepezil in combination, the testing trial time increased compared to the training trial time, indicating that the time to reach the electric shock chamber significantly increased, which indicates that the memory of the mouse was enhanced.

### Experimental Example 2. Morris Water Maze Test

The cognitive function of the mice was checked 3 months after feeding.

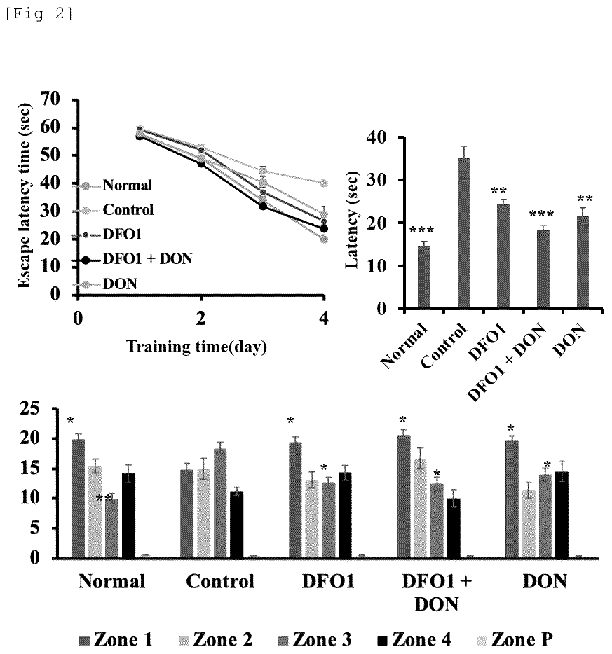

A circular water tank (diameter: 180 cm, height: 65 cm) was filled with water at a temperature of 22±2° C. to a depth of 45 cm, and ink was added to make the water milky white. A platform (diameter: 4.5 cm, height: 43.5 cm) was placed in the center of one of the four fan-shaped sections (Zone 1) of the entire water tank, and the top of the platform was positioned 1.0 cm under the water surface. The training trial was conducted three times a day for four consecutive days. Once the mouse found the platform, it was allowed to remain on the platform for about 10 seconds and then returned to the home cage. The next training trial was conducted 60 minutes later. If the mouse failed to find the platform within 60 seconds, the mouse was allowed to remain on the platform for 10 seconds and then returned to the home cage, and the next training was performed 60 minutes later. A probe trial was conducted on the trained mice 24 hours after the last training trial. The probe trial was conducted by removing the platform from the water tank and measuring the time (latency) spent in each quadrant for 60 seconds. The experimental results were recorded and analyzed using the SMART program (Pan Lab Co., Barcelona, Spain) installed on the ceiling above the water tank, and the results are shown in FIG. 2.

As shown in FIG. 2, it was confirmed that, in the lactic acid bacteria-fermented mulberry fruit extract group (DFO1) fed the lactic acid bacteria-fermented mulberry fruit extract alone and the experimental group (DF01+DON) fed the lactic acid bacteria-fermented mulberry fruit extract and donepezil in combination, the time to find the platform after training significantly decreased compared to that in the control group.

In addition, it was shown that, in the lactic acid bacteria-fermented mulberry fruit extract group (DFO1) fed the lactic acid bacteria-fermented mulberry fruit extract alone and the experimental group (DF01+DON) fed the lactic acid bacteria-fermented mulberry fruit extract and donepezil in combination, when the platform was removed, the time spent in Zone 1, where the platform was located, increased compared to that in the control group, and the time spent in Zone 3, located opposite the platform, decreased to a level similar to that in the normal group, indicating that the cognitive function of the mice was enhanced.

### Experimental Example 3. Y-Maze Test

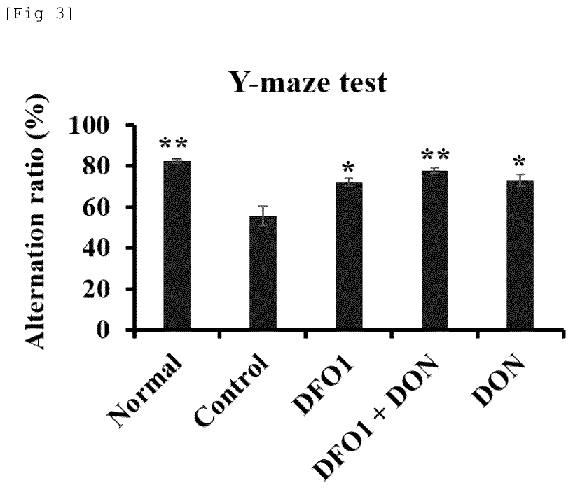

The Y-shaped maze consists of three arms. Each arm was 50 cm in length, 10 cm in width, and 5 cm in height, and the angle between the arms was 120°. The arms were designated as A, B, and C, respectively, and each mouse was carefully placed at the end of one arm. Then, the location to which the mouse moved for 8 minutes was recorded and consecutive entry into the three arms was photographed. A score of 1 point was given only when entering A, B and C once in succession, such as A, B and C, or B, C and A, or C, A and B (excluding cases such as A, A and B, or B, B and A, or C, C and C), and the alternation ratio (%) of the mouse was measured according to the following Equation 1. The results are shown in FIG. 3.

$$Alternation ratio ( \% ) = actual alternation / maximum alternation * 100 \qquad [ Equation 1 ]$$

$$In Equation 1 above , maximum alternation = total number of entries - 2.$$

As shown in FIG. 3, it was shown that, in the lactic acid bacteria-fermented mulberry fruit extract group (DFO1) fed the lactic acid bacteria-fermented mulberry fruit extract alone and the experimental group (DF01+DON) fed the lactic acid bacteria-fermented mulberry fruit extract and donepezil in combination, the alternation ratio increased compared to that in the control group and was recovered to a level similar to that in the normal group. These results suggest that the mice of the lactic acid bacteria-fermented mulberry fruit extract group (DFO1) fed the lactic acid bacteria-fermented mulberry fruit extract alone and the experimental group (DF01+DON) fed the lactic acid bacteria-fermented mulberry fruit extract and donepezil in combination had enhanced memory.

### Experimental Example 4. Western Blot Analysis

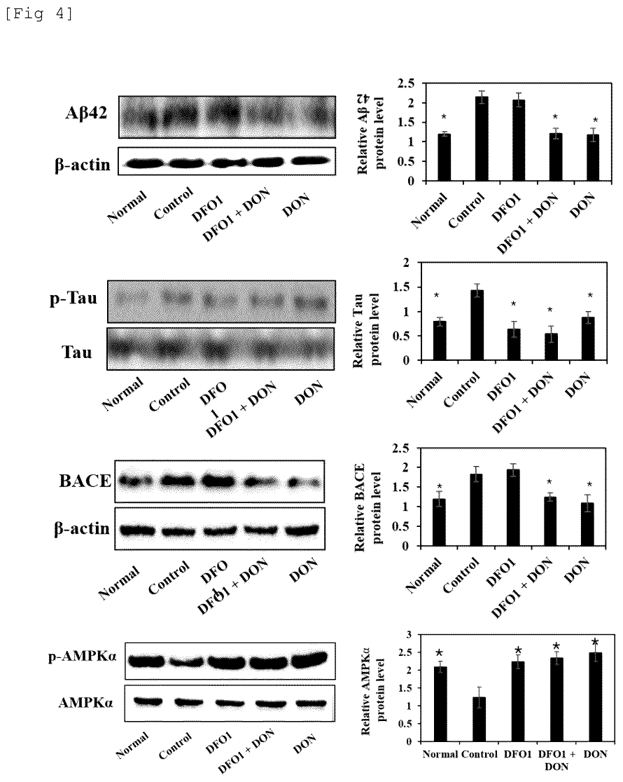

After completion of the experiment, the mouse brain was harvested and homogenized in buffer (50 mM Tris-HCl pH 7.5, 2 mM EDTA, 150 mM NaCl, 30 mM sodium pyrophosphate, 2 mM Na₃VO₄, 10 mM NaF, protease inhibitor cocktail). Then, the homogenized tissue was centrifuged at 12,000 rpm for 15 minutes at 4° C. and the supernatant was collected. Protein quantification was performed using the bicinchoninic acid (BCA, Pierce) method. The supernatant was added to 4×Lammlie buffer (10% 2-mercaptoethanol, 62.5 mmol/L Tris-HCl, pH 6.8, 20% glycerol, 2% SDS), followed by boiling at 99° C. for 5 minutes. 30 μg of the quantified protein sample was separated by electrophoresis (SDS-PAGE) and transferred to a nitrocellulose paper (membrane). Then, the membrane was washed three times with Tris-buffered saline (TBST) containing 0.1% Tween 20, and blocked in 5% nonfat dry milk for 30 minutes or more. Next, primary antibodies, including Aβ42, BACE, β-actin, phosphorylated Tau (p-Tau), Tau, phosphorylated AMPKα (p-AMPKα), and AMPKα, were added to the membrane which was then incubated at 4° C. for 12 hours or more, washed four times with TBST for 15 minutes each. Next, the membrane was incubated with secondary antibodies for 2 hours. The membrane was then washed four times and the proteins were visualized using an enhanced chemiluminiscence system (ECL, Pierce). The quantitative analysis of the proteins was performed using an imaging system (LAS-3000, Fuji). The results are shown in FIG. 4.

As shown in FIG. 4, Aβ42, p-Tau, and BACE significantly increased in the control group compared to the normal group, and p-AMPKα significantly decreased in the control group compared to the normal group.

On the other hand, it was confirmed that, in the lactic acid bacteria-fermented mulberry fruit extract group (DFO1) fed the lactic acid bacteria-fermented mulberry fruit extract alone, the expression level of p-Tau decreased compared to that in the control group and was similar to that in the normal group, and the expression level of p-AMPKα increased and was similar to that in the normal group.

In addition, it was confirmed that, in the experimental group (DF01+DON) fed the lactic acid bacteria-fermented mulberry fruit extract and donepezil in combination, the expression levels of Aβ42, p-Tau and BACE decreased compared to those in the control group and were similar to those in the normal group, and the expression level of p-AMPKα increased and was similar to that in the normal group.

These results suggest that administration of the lactic acid bacteria-fermented mulberry fruit extract alone (DFO1) or co-administration of the lactic acid bacteria-fermented mulberry fruit extract and donepezil (DF01+DON) has an anti-Alzheimer's disease effect.

### Experimental Example 5. Evaluation of Neuroprotective Effect

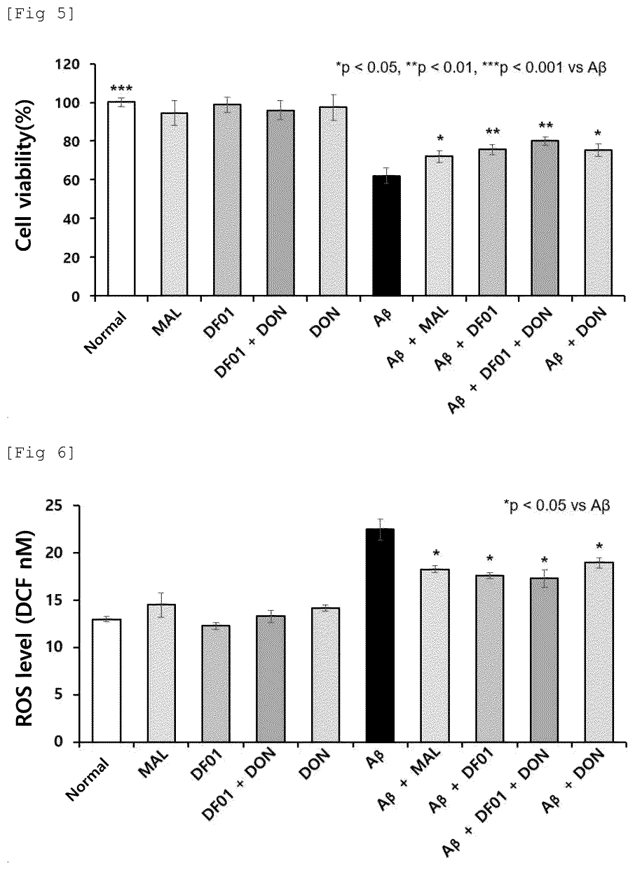

To evaluate the effect of protecting against neuronal damage caused by amyloid-beta (Aβ), a neuronal cell line (SH-SY5Y cells) and a neuronal cell line (SH-SY5Y cells) treated with amyloid-beta (Aβ, 25 μM) were treated with each of the mulberry fruit extract (MAL, 200 μg/mL), the lactic acid bacteria-fermented mulberry fruit extract (DFO1, 200 μg/mL), donepezil (DON, 5 μM), and the lactic acid bacteria-fermented mulberry fruit extract plus donepezil (DF01 (200 μg/mL)+DON (5 μM)). The cell viability of the neuronal cell lines (SH-SY5Y cells) was measured, and the results are shown in FIG. 5.

As shown in FIG. 5, it was confirmed that the group treated with the lactic acid bacteria-fermented mulberry fruit extract alone (DFO1) showed a cell viability close to 100% in the same manner as the untreated normal group.

On the other hand, it was confirmed that the group treated with the mulberry fruit extract alone (MAL) showed a slightly lower cell viability than the normal group and the group treated with the lactic acid bacteria-fermented mulberry fruit extract alone (DFO1).

In addition, referring to the experimental results of amyloid beta (Aβ) treatment in FIG. 5, it was confirmed that the group treated with the lactic acid bacteria-fermented mulberry fruit extract alone (Aβ+DF01) showed a higher cell viability than the group treated with the mulberry fruit extract alone (Aβ+MAL), and thus had a better neuroprotective effect. In particular, the group treated with the lactic acid bacteria-fermented mulberry fruit extract alone (Aβ+DF01) was “**”, and the group treated with the mulberry fruit extract alone (Aβ+MAL) was “*”, suggesting that administration of the lactic acid bacteria-fermented mulberry fruit extract alone (Aβ+DF01) has a more significant neuroprotective effect.

In addition, it was confirmed that co-administration of the lactic acid bacteria-fermented mulberry fruit extract and donepezil (Aβ+DF01+DON) had a better neuroprotective effect than administration of the lactic acid bacteria-fermented mulberry fruit extract alone (Aβ+DF01) or the mulberry fruit extract alone (Aβ+MAL).

### Experimental Example 6. Evaluation of Antioxidant Effect

To evaluate the antioxidant effect of protecting against oxidation caused by amyloid-beta (Aβ), in the same manner in Experimental Example 5 above, a neuronal cell line (SH-SY5Y cells) and a neuronal cell line (SH-SY5Y cells) treated with amyloid-beta (AR, 25 μM) were treated with each of the mulberry fruit extract (MAL, 200 μg/mL), the lactic acid bacteria-fermented mulberry fruit extract (DFO1, 200 μg/mL), donepezil (DON, 5 μM), and the lactic acid bacteria-fermented mulberry fruit extract plus donepezil (DF01 (200 μg/mL)+DON (5 μM)). The reactive oxygen species (ROS) levels of the neuronal cell lines (SH-SY5Y cells) were measured, and the results are shown in FIG. 6.

As shown in FIG. 6, it was confirmed that the group treated with the lactic acid bacteria-fermented mulberry fruit extract alone (DFO1) showed a lower reactive oxygen species level than the untreated normal group, but the group treated with the mulberry fruit extract alone (MAL) showed a higher reactive oxygen species level than the normal group, suggesting that administration of the lactic acid bacteria-fermented mulberry fruit extract alone (DFO1) has a better antioxidant effect.

In addition, referring to the experimental results of amyloid beta (Aβ) treatment in FIG. 6, it can be seen that administration of the lactic acid bacteria-fermented mulberry fruit extract alone (Aβ+DF01) has a better antioxidant effect than administration of the mulberry fruit extract alone (Aβ+MAL).

Furthermore, it was confirmed that co-administration of the lactic acid bacteria-fermented mulberry fruit extract and donepezil (Aβ+DF01+DON) had a better antioxidant effect than administration of the lactic acid bacteria-fermented mulberry fruit extract alone (Aβ+DF01) or donepezil alone (Aβ+DON).

Although the present invention has been described above with reference to the limited embodiments, the present invention is not limited by these embodiments, and those skilled in the art to which the present invention pertains will appreciate that various modifications and variations are possible without departing from the technical idea of the present invention and the equivalent scope of the appended claims.

1. A method for preventing or treating Alzheimer's disease in a subject in need thereof, the method comprising administering to the subject a composition comprising a mulberry fruit extract fermented with lactic acid bacteria as an active ingredient.

2. The method for preventing or treating Alzheimer's disease according to claim 1, wherein the mulberry fruit extract is obtained by extraction with at least one method selected from hot-water extraction and ultrasonic extraction.

3. The method for preventing or treating Alzheimer's disease according to claim 1, wherein the mulberry fruit extract is obtained by extraction with at least one solvent selected from water and a C₁-C₄ alcohol.

4. The method for preventing or treating Alzheimer's disease according to claim 1, wherein the lactic acid bacteria are at least one selected from Lactobacillus brevis DF01 and Pediococcus acidilactici K10.

5. The method for preventing or treating Alzheimer's disease according to claim 1, wherein the mulberry fruit extract fermented with lactic acid bacteria is obtained by inoculating and fermenting 100 parts by weight of the mulberry fruit extract with 0.1 to 5 parts by weight of the lactic acid bacteria.

6. The method for preventing or treating Alzheimer's disease according to claim 1, wherein the mulberry fruit extract fermented with lactic acid bacteria is obtained by fermentation at 20° C. to 30° C. for 2 days to 5 days.

7. The method for preventing or treating Alzheimer's disease according to claim 1, wherein the composition further comprises donepezil.

8. A method for preventing or alleviating Alzheimer's disease in a subject in need thereof, the method comprising administering to the subject of a food composition comprising a mulberry fruit extract fermented with lactic acid bacteria.
